(19) United States
(12) Patent Application Publication
(10) Pub. No.: US 2009/0321349 A1
(43) Pub. Date: Dec. 31, 2009

(54) Integrated systems for producing biogas and liquid fuel from algae

(76) Inventors: John D. Offerman, Orono, MN (US); Hugh Mc Tavish, Birchwood, MN (US)

Correspondence Address: HUGH MCTAVISH;MCTAVISH PATENT FIRM, 429 BIRCHWOOD COURTS, BIRCHWOOD, MN, 55110, US

(21) Appl. No.: 12/459,136

(22) Filed: Jun. 26, 2009

Related U.S. Application Data
(60) Provisional application No. 61/133,198, filed on Jun. 26, 2008.

(51) Int. Cl. C12M 1/107

(52) U.S. Cl. 210/603; 435/292.1

(57) ABSTRACT

The embodiments of the invention provide methods and systems for making biogas by growth of aquatic plants followed by anaerobic digestion of organic material from the aquatic plants to biogas comprising methane and carbon dioxide. The biogas can be burned as a fuel or can optionally be further processed to produce a liquid fuel, typically alcohol or diesel, by a Fischer-Tropsch process. The biogas is converted to synthesis gas comprising CO and H₂, and the synthesis gas is contacted with a catalyst to be converted to liquid fuels.

This application claims priority under 35 U.S.C. 119(e) from U.S. provisional patent application Ser. No. 61/133,198, filed Jun. 26, 2008.

### BACKGROUND

The world increasingly faces energy shortages as oil prices have risen to well over $100 per barrel. In addition, the burning of fossil fuels produces carbon dioxide, which is primarily responsible for the greenhouse effect and threatens to alter the climate to an extent that threatens to cause increased storms, flooding of coastal areas and the disappearance of some island nations, drought and famine in adversely affected areas, and damage to many ecosystems and extinction of species.

New sources of fuel and energy are needed, especially renewable energy that does not contribute to greenhouse warming.

### SUMMARY

The embodiments of the invention provide methods and systems for making biogas by growth of aquatic plants followed by anaerobic digestion of organic material from the aquatic plants to biogas comprising methane and carbon dioxide. The biogas can be burned as a fuel or can optionally be further processed to produce a liquid fuel, typically alcohol or diesel, by a Fischer-Tropsch process. The biogas is converted to synthesis gas comprising CO and H₂, and the synthesis gas is contacted with a catalyst to be converted to liquid fuels.

One embodiment of the invention provides a method of producing liquid fuel comprising: (a) cultivating aquatic plants in an aquatic medium exposed to light in a photosynthetic culture chamber; (b) transferring organic material of the aquatic plants to an anaerobic digester; (c) fermenting the organic material in the digester to produce a biogas comprising methane and carbon dioxide; (d) converting at least a portion of the biogas to synthesis gas comprising CO and H₂; and (e) contacting at least a portion of the synthesis gas with a catalyst to produce a liquid fuel. The liquid fuel may be, for instance, methanol, ethanol, higher alcohols, or diesel.

Another embodiment of the invention provides a method of producing biogas comprising: (a) cultivating aquatic plants in an aquatic medium exposed to light in a photosynthetic culture chamber; (b) transferring organic material of the aquatic plants to an anaerobic digester; (c) fermenting the organic material in the digester to produce a liquid effluent and a biogas comprising methane and carbon dioxide; and (d) transferring CO₂ to the aquatic medium in the photosynthetic culture chamber by one of three methods. CO₂ can be transferred to the aquatic medium in the photosynthetic culture chamber by transferring at least a portion of the liquid effluent from the anaerobic digester to the photosynthetic culture chamber wherein the liquid effluent has a concentration of dissolved CO₂ of at least 3 mM when contacted with the aquatic medium in the photosynthetic culture chamber. CO₂ can also be transferred by contacting at least a portion of the biogas with an aqueous volume outside of the anaerobic digester and the photosynthetic culture chamber to dissolve CO₂ from the biogas in the aqueous volume and generate a CO₂-depleted biogas, and mixing the aqueous volume with the aquatic medium in the photosynthetic culture chamber. CO₂ can also be transferred by contacting at least a portion of the biogas with the aquatic medium in the photosynthetic culture chamber to dissolve CO₂ from the biogas in the aquatic medium and generate a CO₂-depleted biogas.

Another embodiment of the invention provides a system for producing liquid fuel comprising: (a) a photosynthetic culture chamber for culturing aquatic plants in an aquatic medium exposed to light in the photosynthetic culture chamber; (a) being functionally coupled to (b) an anaerobic digester for producing biogas comprising CH₄ and CO₂ from organic material of the aquatic plants in an anaerobic digestion mixture in the anaerobic digester; (b) being functionally coupled to (c) a device for producing synthesis gas comprising CO and H₂ from biogas comprising CH₄, the device comprising a steam reformer, a CO₂ reformer, a partial oxidation unit, or a combination thereof; (c) being functionally coupled to (d) a catalyst for converting synthesis gas to a liquid fuel.

Another embodiment of the invention provides a system for producing biogas comprising: (a) a photosynthetic culture chamber for culturing aquatic plants in an aquatic medium exposed to light in the photosynthetic culture chamber; (a) being functionally coupled to (b) an anaerobic digester for producing biogas comprising CH₄ and CO₂ from organic material of the aquatic plants in an anaerobic digestion mixture in the anaerobic digester; (b) being functionally coupled to (c) a conduit functionally coupled to (a) and (b) and adapted for transferring liquid effluent from the anaerobic digester to aquatic medium in the photosynthetic culture chamber without equilibration with air before the liquid effluent is mixed with the aquatic medium in the photosynthetic culture chamber.

Another embodiment of the invention provides a system for producing biogas comprising: (a) a photosynthetic culture chamber for culturing aquatic plants in an aquatic medium exposed to light in the photosynthetic culture chamber; (a) being functionally coupled to (b) an anaerobic digester for producing biogas comprising CH₄ from organic material of the aquatic plants in an anaerobic digestion mixture in the anaerobic digester; (c) a chamber for holding an aqueous volume separate from the anaerobic digester and the photosynthetic culture chamber; (d) a conduit functionally coupled to (b) and (c) and adapted to transfer biogas from the anaerobic digester (b) to (c) and contact the biogas with the aqueous volume in the chamber (c) to dissolve CO₂ from the biogas in the aqueous volume in (c); the chamber (c) functionally coupled to (e) a conduit functionally coupled to (c) and (a) and adapted to transfer the aqueous volume to aquatic medium in the photosynthetic culture chamber without equilibration with air before the aqueous volume is mixed with the aquatic medium in the photosynthetic culture chamber.

Another embodiment of the invention provides a system for producing biogas comprising: (a) a photosynthetic culture chamber for culturing aquatic plants in an aquatic medium exposed to light in the photosynthetic culture chamber; (a) being functionally coupled to (b) an anaerobic digester for producing biogas comprising CH₄ and CO₂ from organic material of the aquatic plants in an anaerobic digestion mixture in the anaerobic digester; (b) being functionally coupled to (c) a conduit functionally coupled to the anaerobic digester and the photosynthetic culture chamber and adapted to transfer at least a portion of the biogas from the anaerobic digester to the photosynthetic culture chamber to contact the at least a portion of the biogas with the aquatic medium in the photosynthetic culture chamber to dissolve CO₂ from the biogas in the aquatic medium and generate a CO₂-depleted biogas.

### BRIEF DESCRIPTION OF THE DRAWINGS

FIG. 1 is a schematic drawing of a system for producing biogas or liquid fuel.

FIG. 2 is a schematic drawing of another system for producing biogas.

FIG. 3 is a schematic drawing of another system for producing biogas.

### DETAILED DESCRIPTION

Different embodiments of the invention provide methods of making biogas or liquid fuel and systems adapted to carry out the methods. A liquid fuel, as used herein, is a reduced carbon substance that is liquid at room temperature and atmospheric pressure.

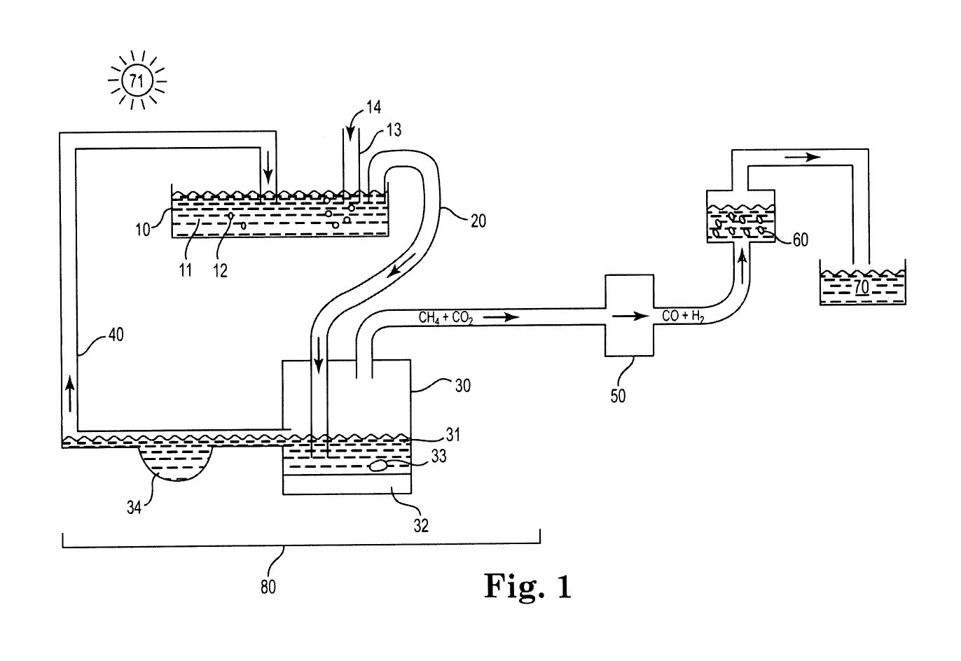

One system of the invention is shown in FIG. 1. FIG. 1 is a schematic drawing of a system of the invention for producing liquid fuel. FIG. 1 shows a photosynthetic culture chamber 10 for culturing aquatic plants 12 in an aquatic medium 11 exposed to light in the photosynthetic culture chamber 10. The light source is shown as the sun 71. Artificial light can also be used but obviously that requires the consumption of energy. The photosynthetic culture chamber 10 is linked to anaerobic digester 30 by conduit 20 that transfers organic matter from the photosynthetic culture chamber 10 to anaerobic digester 30. In one embodiment, the aquatic medium 11 containing aquatic plants 12 is transferred directly to the anaerobic digester 30 without separating aquatic plants 12 from the aquatic medium of the photosynthetic culture chamber (i.e., without harvesting and concentrating the aquatic plants).

In one embodiment, the aquatic plants are planktonic algae suspended in the photosynthetic culture medium.

The anaerobic digester 30 contains organic material 32 of the aquatic plants in fermentation mixture 31 digested by microorganisms 33. The digestion produces a biogas comprising CH₄. The biogas comprising methane is passed to a device 50 for producing synthesis gas comprising CO and H₂ from biogas. The device 50 can include a steam reformer, partial oxidation unit, or CO₂ reformer, or a combination thereof. CO and H₂ exit device 50 and pass to catalyst 60, which converts synthesis gas to a liquid fuel 70. The catalyst 60 catalyzes formation of typically an alcohol and/or diesel fuel. The alcohol may be methanol, ethanol, or higher alcohols (C₃+ alcohols, i.e., those containing 3 carbons or more). Diesel fuel may also be formed by catalyst 60.

Formation of methanol and alkanes (a component of diesel fuel) by the Fischer-Tropsch reaction on catalyst 60 is by the following reactions.

1CO+2H₂-->CH₃OH

(n+2)CO+(2n+5)H₂-->CH₃(CH₂)ₙCH₃+(n+2)H₂O

Ethanol, higher alcohols, ketones, aldehydes, and other liquid fuel organic compounds can be formed by similar reactions from synthesis gas.

In one embodiment of the invention, a combustion exhaust gas, for example from a fossil fuel power plant, is bubbled into the aquatic medium of the photosynthetic culture chamber to add CO₂ to the aquatic medium. If algae or other aquatic plants have adequate nutrients such as nitrogen and phosphorous, and adequate light, the limiting factor in photosynthetic productivity is CO₂. Providing additional CO₂ to the aquatic medium of the photosynthetic culture chamber can greatly enhance photosynthetic yield. Regan et al. (Regan, D. L. and Ivancic N., Biotechnology and Bioengineering 26:1265-1271 (1984)) reported that when mixed microalgae cultures grown in medium enriched with nitrogen and phosphorous were sparged with 1% CO₂, productivity of the cultures increased more than 4-fold over cultures grown in enriched medium without CO₂ sparging.

Accordingly, FIG. 1 shows sparger 13 delivering a CO₂-rich combustion exhaust gas 14 to the aquatic medium 11 in the photosynthetic culture chamber 10.

FIG. 1 also shows a conduit 40 for transferring effluent 34 from anaerobic digester 30 to photosynthetic culture chamber 10. The conduit 40 is adapted to transfer effluent 34 without allowing the liquid effluent 34 to equilibrate with air. Biogas comprises CO₂ as well as CH₄. Biogas is typically approximately 40% CO₂ and 60% CH₄. Thus, the biogas has a high CO₂ concentration, and this dissolves in the liquid medium in the anaerobic digester. Liquid effluent from the anaerobic digester thus has a high dissolved CO₂ concentration when it leaves the anaerobic digester. It thus can be used as a CO₂ supplement for the aquatic medium of the photosynthetic chamber. But it should be transferred to mix with the aquatic medium of the photosynthetic chamber in a manner such that it retains its high CO₂ concentration and does not equilibrate with air. In preferred embodiments, the CO₂ concentration of the effluent is at least 3 mM at the time it is mixed with the aquatic medium of the photosynthetic culture chamber. In other embodiments, the concentration of CO₂ in the liquid effluent is at least 1 mM, at least 5 mM, or at least 10 mM at the time it is mixed with the aquatic medium of the photosynthetic culture chamber. The concentration of CO₂ in water equilibrated with CO₂ at 1 atm pressure is 39 mM.

The effluent can be a solid or a liquid effluent, or a combination of both.

FIG. 1 also shows a system 80 for producing biogas comprising: (a) a photosynthetic culture chamber 10 for culturing aquatic plants in an aquatic medium exposed to light in the photosynthetic culture chamber 10; (a) being functionally coupled to (b) an anaerobic digester 30 for producing biogas comprising CH₄ and CO₂ from organic material of the aquatic plants in an anaerobic digestion mixture in the anaerobic digester; (b) being functionally coupled to (c) a conduit 40 functionally coupled to (a) and (b) and adapted for transferring liquid effluent 34 from the anaerobic digester 30 to aquatic medium 11 in the photosynthetic culture chamber 10 without equilibration with air before the liquid effluent is mixed with the aquatic medium in the photosynthetic culture chamber.

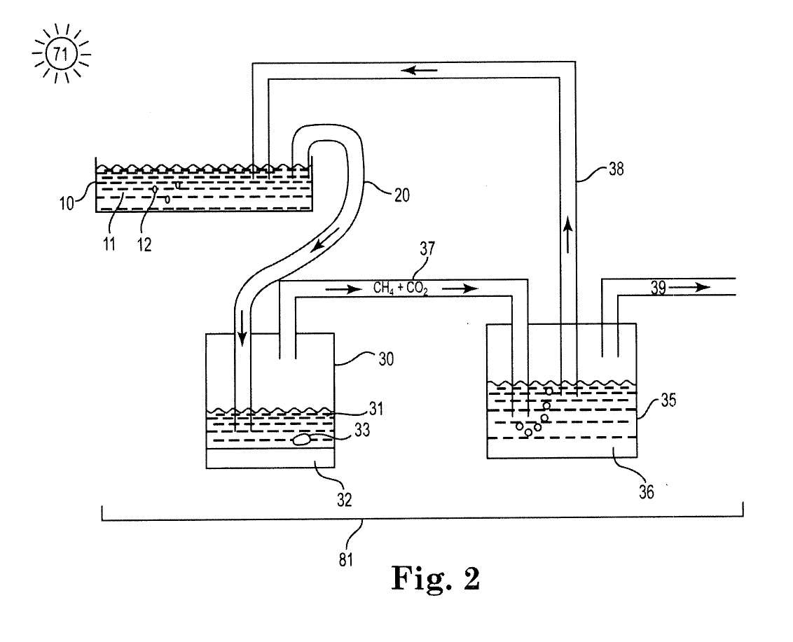

FIG. 2 shows another system for producing biogas. Digester 30 includes microorganisms 33 that ferment organic material 32 of the aquatic plants 12 to biogas. The system includes a chamber 35 for holding an aqueous volume 36. Biogas comprising CO₂ and CH₄ is transferred from anaerobic digester 30 via conduit 37 to the aqueous volume 36 in chamber 35. The biogas contacts the aqueous volume to dissolve CO₂ in the aqueous volume 36, and then the aqueous volume is transferred to the aquatic medium 11 in photosynthetic chamber 10 via conduit 38. After contacting with the aqueous volume 36 in chamber 35, CO₂-depleted biogas 39 is produced and can be harvested for use to produce liquid fuels, for combustion to provide energy, or for other uses.

Biogas comprises CO₂ and CH₄ predominantly. Carbon dioxide is significantly more soluble in water than methane. Thus, by sparging or otherwise contacting the biogas with an aqueous volume, CO₂ is preferentially dissolved over CH₄. Thus, the aqueous volume becomes enriched in CO₂ but has relatively little dissolved methane, while the gas phase becomes depleted in CO₂ but has relatively little loss of methane. In some embodiments, aqueous volume 36 includes carbonic anhydrase, which catalyzes conversion of dissolved CO₂ to H₂CO₃. That can enhance dissolution of CO₂ by removing it from solution. In addition, bicarbonate is often the biologically used substrate, rather than CO₂. The carbonic anhydrase optionally can be immobilized to a solid substrate so that it can be reused.

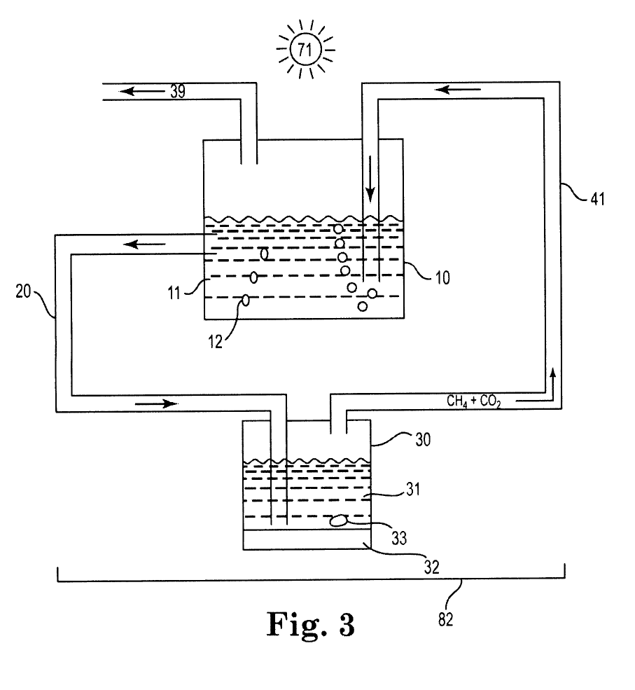

FIG. 3 shows another system of the invention. In FIG. 3 conduit 41 couples the anaerobic digester to the photosynthetic culture chamber and transfers at least a portion of the biogas from the anaerobic digester to contact the aquatic medium in the photosynthetic culture chamber (e.g., by sparging the biogas into the aquatic medium) to dissolve CO₂ from the biogas in the aquatic medium. This transfers CO₂ from the biogas to the aquatic medium to supplement the medium in CO₂ to improve plant growth. It also depletes the biogas of CO₂ and generates a CO₂-depleted biogas 39 that can be collected. The aquatic medium optionally may include carbonic anhydrase.

In one embodiment, the aquatic plants are planktonic. The term “aquatic plants” as used herein refers to organisms that perform oxygenic photosynthesis and are at least partially submerged in standing or flowing water. Planktonic aquatic plants are small plants that float or drift in water. In one embodiment, the planktonic plants are microalgae. Examples of commonly cultivated microalgae genera include Chlorella, Synechocystis, Nostoc, and Spirulina. The planktonic plants can also be macroscopic, such as Lemna (duckweed). A pure culture is not necessary. Thus, in one preferred embodiment, a mixture of microalgae species are placed in the photosynthetic culture chamber and allowed to compete to select for the best strain under the conditions used. In another preferred embodiment, microalgae are allowed to colonize the photosynthetic culture chamber by drift of airborne particles into the chamber or by seeding the chamber with a sample of a natural body of water containing microalgae, and allowing a dominant strain or mixture of strains to arise spontaneously.

In another embodiment, the aquatic plants are rooted in sediment or adherent to a surface.

In one embodiment, the aquatic plants are water hyacinth.

In one embodiment, the aquatic plants are adherent to a surface. Harvesting these can involve scraping a fraction of the plant material from the surface, while leaving a large fraction as starting material for further growth. An example of this is described in Bayless, U.S. publish patent application no. 20020072109.

In one embodiment, the aquatic plants are planktonic and the step of transferring organic material of the aquatic plants to an anaerobic digester comprises transferring the aquatic medium containing the aquatic plants to the digester, without separating the aquatic plants from the aquatic medium.

Harvesting is one of the most expensive aspects of growing algae. Harvesting usually involves concentrating algae, which is to say separating them from the aquatic medium. This may be accomplished by biological flocculation, chemical flocculation, or straining through a screen. Biological flocculation refers to inducing algae to flocculate (adhere to other algal cells or organisms to form larger clumps) and settle spontaneously by natural means. For instance, some algae, if grown with significant fluid shear, flocculate when the fluid movement is stopped. Chemical flocculation refers to adding chemicals to the aquatic medium to induce the algae to flocculate. Straining through a screen is another commonly used method to harvest microalgae. One difficulty with straining is that smaller colonies will pass through the screen and remain in the medium, so that smaller colonies are selected for and gradually can dominate the culture. (Benemann, J. R. pp. 317-337, in Algal and Cyanobacterial Biotechnology, 1989, Longman Scientific and Technical, Essex, UK.)

Since harvesting algae, in the sense of separating the algae from the aquatic medium, can be an expensive step, it can be advantageous to eliminate this step by simply transferring aquatic medium containing the aquatic plants to the digester, without separating the aquatic plants from the aquatic medium. One concern when this technique is used is that the photosynthetic aquatic medium contains significant amounts of dissolved oxygen. Oxygen is toxic to methanogens. Thus, it is typically important to remove most or all oxygen from the additions to a methane-producing digester. For this reason and others, in some embodiments it may be advantageous for the anaerobic digester to involve two anaerobic digestion vessels operated in sequence. The first digester vessel may contain predominantly acid-forming bacteria. It may tolerate addition of aerobic fluids. Oxygen in the fluids can be consumed in this digester vessel containing microorganisms that are tolerant of oxygen. Anaerobic effluent from the first digester vessel can be fed into a second digester vessel where most production of methane occurs.

In other embodiments, the aquatic plants are harvested (separated) from the aquatic medium before transferring organic material of the aquatic plants to the anaerobic digester.

In a preferred embodiment, the aquatic plants are transferred as whole aquatic plants to the anaerobic digester. In other embodiments, the aquatic plants are processed before transfer to the anaerobic digester. If the aquatic plants are macroscopic, it may, for instance, speed digestion to grind the aquatic plants into smaller particles before transferring them to the digester.

In some embodiments, the aquatic plants are harvested and then processed to extract valuable products, leaving a biomass residue. The valuable product, in one embodiment, is oil. Many types of microalgae, including diatoms, have high concentrations of oil. The oil can be harvested for use directly, e.g., as diesel fuel, or can be processed further, for instance by transesterification with methanol and sodium hydroxide to form methyl esters of fatty acids, which is a better diesel fuel. Methods of extracting oil from microalgae and other aquatic plants are known to persons of ordinary skill in the art.

After extracting oil or another valuable product from algae or another aquatic plant, the biomass residue may be transferred to the anaerobic digester for digestion to biogas.

The anaerobic digestion step occurs in an anaerobic digester under anaerobic conditions. Fermentation to biogas generally occurs by the action of two types of microorganisms. Acid forming bacteria break down larger organic molecules to organic acids, H₂ and CO₂. The acid-formers are relatively fast growing and tolerant of variations in conditions, including tolerant to O₂. The second step in the fermentation is carried out by methanogens, which convert organic acids or H₂ and CO₂ to methane. The methanogens are slower growing and less tolerant to O₂ and variations in pH. The pH optimum for the methanogens is about pH 6.0 to 7.0. The acid-forming bacteria have a pH optimum above 7.0. Digestion to biogas may be carried out in various digester designs known to persons skilled in the art including batch, continuously stirred tank reactors, plug-flow, attached film and fluidized bed digesters (Chynoweth, D. P. pp. 1-14, in Anaerobic Digestion of Biomass, Elsevier Applied Science, London, 1987. Fannin, K. F. and Biljetina, R, pp. 141-170, in Anaerobic Digestion of Biomass, Elsevier Applied Science, London, 1987.) Other techniques and microorganisms for anaerobic digestion are disclosed in U.S. Pat. No. 7,309,592, which is incorporated by reference.

In some embodiments, the step of fermenting organic material to a biogas involves the steps of feeding the organic material into a vessel, fermenting and mixing the organic material in anaerobic conditions in the vessel to form the biogas, discontinuing the mixing to allow particulate unfermented organic material to settle in the vessel resulting in the formation of a low-suspended-solid supernatant, decanting the supernatant from the vessel, and repeating at least the feeding and fermenting steps. This method improves the efficiency of gasification of the organic material, as compared to a two-vessel system (U.S. Pat. No. 5,185,079). In the two-vessel system, fermentation occurs in one vessel and then the wastewater flows to a separate solids separation unit where settling takes place. The settled solids are then recycled to the fermentation vessel (U.S. Pat. No. 5,185,079). The one-vessel system also requires less capital investment.

In one embodiment of the one-vessel method, the method further involves withdrawing at least a portion of the biogas from the vessel, optionally with the use of a vacuum, immediately before the settling step.

In one embodiment of the invention, the step of fermenting produces an effluent; and the method comprises transferring at least a portion of the effluent from the anaerobic digester to the photosynthetic culture chamber. The effluent is typically rich in nutrients, including nitrogen, phosphorous, and potassium that are essential for the photosynthetic organisms. Thus, recirculating the effluent from the anaerobic digester to the photosynthetic culture chamber can reduce or eliminate the need to supplement the aquatic medium of the photosynthetic culture chamber with certain nutrients.

By recirculating effluent from the anaerobic digester to the photosynthetic culture chamber, the whole system for producing biogas or liquid fuel can be almost completely self-contained with very little input or waste product. Photosynthesis splits water to generate oxygen gas and reducing equivalents that are used to reduce carbon dioxide to reduced carbon compounds. Thus, water is consumed in the process and water input is necessary. But potentially the only inputs are water and sunlight, and the only outputs are biogas or liquid fuel. All reduced carbon in the anaerobic digester can come from photosynthetic plants produced in the photosynthetic culture chamber. The nutrients needed for both the aquatic plants and the microorganisms in the anaerobic digester can be recycled between the photosynthetic culture chamber and the anaerobic digester.

Thus, in one embodiment, all reduced carbon feed to the anaerobic digester is biomass of the aquatic plants.

In other embodiments, additional reduced carbon feed may be added to the anaerobic digester. For instance, sewage or other biological waste products can be added to the digester. These also may be added to the photosynthetic culture chamber to provide nutrients to the aquatic medium for the aquatic plants.

Nutrients, including for instance biologically available nitrogen, phosphorous, potassium, iron, or vitamins, may be added to the anaerobic digester from a source distinct from the plants or aquatic medium of the photosynthetic culture chamber. Likewise, nutrients may be added to the photosynthetic culture chamber from a source other than effluent of the anaerobic digester. But preferably the need to add these exogenous nutrients is eliminated or reduced.

Liquid effluent initially has a high concentration of CO₂ when it leaves the anaerobic digester, because biogas is typically about 40% CO₂ and the medium of the anaerobic digester is in equilibrium with the biogas. Since CO₂ is often the limiting nutrient for photosynthetic yield, it is advantageous to transfer this effluent to the photosynthetic chamber without allowing the effluent to equilibrate with air and lose too much of its dissolved CO₂. Thus, in one embodiment, the liquid effluent has a concentration of dissolved CO₂ of at least 3 mM when contacted with the aquatic medium in the photosynthetic culture chamber. In other embodiments, it has a concentration of dissolved CO₂ of at least 1 mM, at least 5 mM, at least 7 mM, or at least 10 mM when contacted with the aquatic medium in the photosynthetic culture chamber. The concentration of dissolved CO₂ in equilibrium with a 100% CO₂ atmosphere at 1 atmosphere pressure is about 39 mM. CO₂ in aqueous solution eventually equilibrates with carbonate species. But carbonate, bicarbonate, and carbonic acid are not included in the concentration of dissolved CO₂ as defined herein.

In another embodiment of the methods, the methods involve transferring the effluent from the anaerobic digester to a treatment chamber, treating the effluent in the treatment chamber to generate a treated effluent, and transferring the treated effluent to the photosynthetic culture chamber. Treatment might involve, for instance, adjusting the pH, adding nutrients, or removing or destroying certain substances that are harmful to the photosynthetic organisms.

In one embodiment of the methods of producing biogas or liquid fuel the method includes: feeding a CO₂-rich combustion exhaust gas fraction from a hydrocarbon fuel combustion process to the photosynthetic culture chamber. If light and nutrients are sufficient, carbon is the limiting factor in photosynthetic productivity. Sparging with 1% CO₂ was reported to improve microalgae photosynthetic yield over 4-fold (Regan, D. L. and Ivancic N., Biotechnology and Bioengineering 26:1265-1271 (1984)). Thus, feeding the combustion exhaust gas to the photosynthetic culture improves the yield of the culture.

One embodiment of the methods of the invention involves separating CO₂ from the biogas; and delivering separated CO₂ from the biogas to the aquatic medium in the photosynthetic culture chamber to enrich the aquatic medium in CO₂. CO₂ can be separated from methane in the biogas by several techniques known to persons of skill in the art. Among these is pressure swing adsorption. CO₂ can also be separated from methane by differential solubility in aqueous solutions. CO₂ is much more soluble than methane in aqueous solutions, so filtering biogas through an aqueous volume separates CO₂ from methane by preferentially dissolving CO₂ in the aqueous volume. The aqueous volume then may be transferred to the photosynthetic chamber to add CO₂ to the aquatic medium in the photosynthetic chamber. The aqueous volume may optionally include carbonic anhydrase.

Thus, another embodiment of the invention provides a method involving contacting at least a portion of the biogas with an aqueous volume outside of the anaerobic digester and the photosynthetic culture chamber to dissolve CO₂ from the biogas in the aqueous volume and generate a CO₂-depleted biogas; and mixing the aqueous volume with the aqueous medium in the photosynthetic culture chamber. If the method involves taking the biogas to synthesis gas and then to liquid fuel, the method preferably involves converting at least a portion of the CO₂-depleted biogas to synthesis gas.

Delivery of CO₂ to the aquatic medium of the photosynthetic culture chamber can also be accomplished by sparging biogas into the aquatic medium or otherwise contacting the biogas with the aquatic medium. Thus, one embodiment of the methods involves contacting at least a portion of the biogas with the aquatic medium in the photosynthetic culture chamber to dissolve CO₂ from the biogas in the aquatic medium and generate a CO₂-depleted biogas. If the method involves taking the biogas to synthesis gas and then to liquid fuel, the method preferably involves converting at least a portion of the CO₂-depleted biogas to synthesis gas. However, biogas that passes through the photosynthetic culture chamber aquatic medium will have O₂ in the CO₂-depleted gas. It will also most likely have increased N₂ content. It may be advantageous to remove this O₂, as well as to remove N₂, before converting the CO₂-depleted biogas to synthesis gas or before converting the synthesis gas to liquid fuel. Techniques to remove particular gases from a mixture of gases are known in the art. Appropriate techniques include pressure-swing adsorption, membrane filtration, and distillation (reducing the temperature or increasing the pressure to selectively condense different gases to liquid or solid forms due to differences in their boiling or sublimation points).

The most economical photosynthetic culture chamber is generally open outdoor ponds. One preferred design is a raceway pond, where a paddlewheel provides mixing of the pond. (pp. 63-171, Large Scale Cultivation, in Becker, E. W., Microalgae and Biotechnology, Cambridge University Press, Cambridge, England, 1994). However, when CO₂ is actively added to the aquatic medium by one of the methods described herein, it may be advantageous to partially cover the pond or to use a more enclosed chamber as the photosynthetic culture chamber in order to reduce loss of CO₂. Techniques to retain CO₂, however, must be balanced against the need to release O₂. O₂ is produced by photosynthesis, and if O₂ concentration builds up, it can cause photoinhibition, which is light-induced damage to the photosystems resulting in a decrease in a plant's capacity for photosynthesis (Vonshak, A., Outdoor mass production of Spirulina: the basic concept, pp. 79-99, in Spirulina platensis (Arthrospira): Physiology, Cell-biology, and Biotechnology, 1997, Taylor & Francis, London, UK).

In one embodiment of the invention, the photosynthetic culture chamber is positioned above the anaerobic digester. This allows more efficient use of land area. The anaerobic digester preferably should be in the dark to prevent photosynthetic cells from photosynthesizing and producing oxygen. If sunlight is the source of light for the photosynthetic chamber, then placing the photosynthetic chamber above the anaerobic digester allows more efficient use of land area.

The CO₂ from the biogas can also be used efficiently to produce synthesis gas and to produce liquid fuel from synthesis gas. For instance, CO₂ can be converted to synthesis gas comprising CO and H₂ by a CO₂ reforming reaction of CH₄+CO₂-->2 CO+2H₂. The CO₂ reforming reaction can be used alone or in combination with steam reforming (CH₄+H₂O-->CO+3H₂) and/or partial oxidation (CH₄+½ O₂-->CO+2H₂). CO₂ can also react with H₂ from the synthesis gas to form methanol, which can be further converted to ethanol, higher alcohols, or diesel fuel by the Fischer-Tropsch process. Using CO₂ as well as CH₄ from the biogas in these ways increases the yield of liquid fuel. These points are further elaborated in U.S. patent application Ser. No. 12/074,749, which is incorporated by reference.

U.S. Pat. No. 7,309,592, which is incorporated by reference, also provides further details on catalysts and methods for conversion of syngas to liquid fuel.

Partial oxidation increases the ratio of CO to H₂ in the syngas as compared to steam reforming the methane in biogas. Partial oxidation of methane produces a ratio of 2H₂ per CO. Steam reforming produces a ratio of 3H₂ per CO. The increased CO to H₂ ratio from partial oxidation decreases the amount of methanol and increases the amount of ethanol and higher alcohols produced from the syngas.

CO₂ reforming produces an even lower ratio of H₂ to CO, 1:1. Like partial oxidation, this can be useful to adjust the syngas H₂ to CO ratio to the desired ratio for the desired liquid fuel: production of diesel and higher alcohols is optimize with a higher ratio of CO to H₂ (lower ratio of H₂ to CO) than is needed for production of methanol.

Methods of partial oxidation are well known, and units for partial oxidation of methane to CO and H₂ are commercially available. For instance, partial oxidation can be accomplished by oxygen-starved burning. Steam reformers and CO₂ reformers are commercially available. Steam reforming units can also be used for CO₂ reforming.

CO₂ reforming has the significant advantage that the carbon in the CO₂ molecule can be converted to CO and ultimately included in the liquid fuel. This gives a higher yield of liquid fuel.

One disadvantage of partial oxidation is that it requires a purified oxygen input. Steam reforming can be desirable because it avoids the need for a purified oxygen input.

The CO₂ reforming reaction and steam reforming reaction are both endothermic reactions that are spontaneous in the forward direction only at high temperatures. They thus need an input of heat. Partial oxidation is an exothermic reaction, and it can provide some or all of the heat needed to drive the CO₂ reforming and/or steam reforming reactions where a partial oxidation reaction is used to produce syngas. Where additional heat is needed, or where the partial oxidation reaction is not used, some portion of the biogas can be burned to produce heat to drive the CO₂ reforming and/or steam reforming reactions.

In some embodiments, two or all three of partial oxidation, steam reforming, and CO₂ reforming are combined. Combining the reactions allows one to adjust the H₂:CO ratio in the syngas. If partial oxidation is combined with one or both of steam reforming and CO₂ reforming, the heat from the exothermic partial oxidation reaction can be used to help drive the endothermic steam reforming or CO₂ reforming reactions.

The synthesis gas is then contacted with a Fischer-Tropsch catalyst to produce liquid fuel. Specific catalysts are commercially available for methanol production. Other specific catalysts are commercially available for production of a hydrocarbon mixture that can be refined to diesel, gasoline, jet fuel, or other liquid fuels by refining processes similar to those used with crude oil.

The liquid fuel produced in the process can be methanol, C₂+ alcohols, or diesel, or a combination of two or more of these.

Fuel alcohol preferably is predominantly higher alcohols. Alcohol mixtures that are too rich in methanol are sensitive to phase separation in the presence of water, which is ubiquitous in gasoline systems. Thus, preferably the alcohol products are rich in C₂+ alcohols and have low methanol content. In some embodiments the alcohol comprises less than 5% methanol by weight. Preferably the alcohol comprises at least 70% by weight C₂+ alcohols. In some embodiments, the alcohol comprises less than 0.5% by weight methanol. In some embodiments, the alcohol comprises at least 60% by weight ethanol. In some embodiments the alcohol comprises less than 0.5% by weight methanol and at least 60% by weight ethanol. In some embodiments, the alcohol comprises at least 92.1% by weight ethanol. In some embodiments, the alcohol comprises at least 5% or at least 10% by weight C₃+ alcohols.

Several factors can contribute to obtaining alcohol with a high C₂+ alcohol content. One is use of a syngas having a higher ratio of CO to H₂ (lower ratio of H₂:CO). As discussed above, partial oxidation of methane produces a higher CO:H₂ ratio than steam reforming. Another factor involved in obtaining alcohol with a high C₂+ alcohol content is using a catalyst and reaction conditions that promote C₂+ alcohol formation over methanol formation. Suitable catalysts include the catalysts described in Bao, J. et al., 2003, Chem. Commun. 2003:746-747; U.S. Pat. No. 4,235,801; and U.S. Pat. No. 4,333,852. The catalyst described in Bao et al. is a K—Co—Mo/C catalyst. It is formed by the following procedure. Co(NO₃)₂ and (NH₄)₆Mo₇O₂₄ aqueous solutions are prepared and mixed at a Co/Mo molar ratio of 0.5. Citric acid is added to the solution under constant stirring (citric acid/metalic ions molar ratio=0.1). Then a K₂CO₃ solution is dripped slowly into the solution (K/Mo molar ratio=0.1). The pH value of the solution is adjusted to 3.5 with HCOOH and NH₄OH. The solution is kept in a water bath at 65° C. until the solution becomes a gel. The gel is dried at 120° C. for 15 hours and calcined in argon at 400° C. for 4 hours. Suitable reaction conditions with the synthesis gas are a temperature of 230° C., a pressure of 6.0 MPa, and a gas hour space velocity of 9600 hours⁻¹. Under these conditions, the CO conversion was 7.5%, the alcohol selectivity was 60.4% of carbon, the alcohol space-time yield was 296 g per kg-hour, and the C₂+ alcohol to methanol ratio was 1.48. (Bao, J. et al., 2003, Chem. Commun. 2003:746-747.)

Other suitable catalysts are described in U.S. Pat. No. 4,333,852. The catalysts are ruthenium catalysts with a halogen promoter and a phosphine oxide compound as a solvent. An example of catalyst preparation and alcohol synthesis involves the following procedure. 16 milligrams of Ru atoms as triruthenium dodecacarbonyl, 5.6 mmoles of elemental iodine, and 75 ml of tripropylphosphine oxide are placed in a back-mixed autoclave with a net volume of 128 ml and heated with stirring to 55° C. The reactor is pressurized to 500 psi with CO, heated to 240° C., and then pressurized with a H₂/CO mixture (H₂/CO ratio=2.0) to 6,000 psi. As the reaction proceeds the pressure drops. When it drops to 500 psi, the reactor is repressurized with the synthesis gas to 6,000 psi. With this procedure, ethanol is produced at a rate of 2.05 moles/liter/hour at a selectivity of 50 weight percent. The ethanol plus methanol selectivity is 74 weight percent.

Perhaps the most important mechanism to obtain alcohol with low methanol content and high C₂+ alcohol content is to fractionate the alcohol as it is formed into a C₂+-rich alcohol fraction and a methanol-rich fraction, harvest the C₂+-rich alcohol fraction, and recirculate the methanol-rich fraction into the synthesis gas for contact with the catalyst. Adding methanol to the synthesis gas reaction on the catalyst forces the equilibrium of the CO+2H₂-->CH₃OH reaction to the left (Gavin, D. G. and Richard D. G., European Patent Application 0 253 540). With the equilibrium preventing further net formation of methanol, the CO and H₂ react to form ethanol and other C₂+ products. Recirculated methanol can also be a reactant for formation of C₂+ products by reaction with CO, H₂, and/or a second molecule of methanol. If all methanol produced is recirculated, there is no net production of methanol.

In the methanol-recirculation process, the alcohol products from the alcohol catalyst or catalysts are fractionated into a C₂+-rich alcohol fraction and a methanol-rich fraction. This is preferably done by condensing the C₂+ alcohols from the product mixture at a temperature and pressure below the boiling point of the C₂+ alcohols and above the boiling point of methanol. The gaseous methanol-rich fraction is then mixed with the synthesis gas for contact with the catalyst.

The alcohols produced in the methods of the invention, including the C₂+-rich alcohol fraction separated from the methanol-rich fraction, can be further processed or fractionated. For instance, ethanol can be separated from other alcohols and other components in the mixtures. The mixtures often contain propanol, butanol, and isobutanol, which can be purified. Acetaldehyde, acetic acid, acetic anhydride, and other components may be present in the alcohol mixtures and can be purified or separated from the alcohols.

In some embodiments of the invention, the catalyst is a sulfided, nanosized transition metal catalyst selected from Group VI metals. In some embodiments, the catalyst is a sulfided, nanosized molybdenum catalyst. (U.S. Pat. No. 6,248,796.)

In some embodiments, the sulfided, nanosized transition metal catalyst is suspended in a solvent, e.g., heavy machine oil, and the synthesis gas is contacted with the catalyst at a temperature in the range of 250-325° C. and at a pressure in the range of 500 to 3000 psi.

The catalyst can also be other metal or inorganic catalysts, such as are disclosed in U.S. Pat. Nos. 4,675,344; 4,749,724; 4,752,622; 4,752,623; and 4,762,858.

Preferably, the catalyst is sulfur-free, because a sulfur-containing catalyst leaches sulfur into the alcohol mixtures it produces. Sulfhydryls are undesirable in fuel alcohol because they carry an odor, upon burning they produce sulfur oxides that cause acid rain and human health problems, and they can damage engine parts in internal combustion engines. Thus, preferably the alcohols contain less than 10 ppm sulfur atoms, more preferably less than 1 ppm sulfur atoms. This can be achieved by removing sulfhydryls from biogas before the biogas is converted to synthesis gas, and then using a sulfur-free catalyst for conversion of synthesis gas to alcohol. One method to remove sulfhydryls from biogas is to contact the biogas with a metal cation that binds sulfhydryls, such as Fe²⁺. Another method is to contact the biogas with another type of agent that binds sulfhydryls, such as amine compounds, which may be immobilized on a resin.

Alternatively, sulfhydryls can be removed from the alcohol product. One method to do this is to contact the alcohol with a metal cation that binds sulfhydryls, such as Fe²⁺. Another method is to contact the alcohol with another type of agent that binds sulfhydryls, such as amine compounds, which may be immobilized on a resin.

In particular embodiments of the methods and products of the invention, the alcohol or purified alcohol has less than 10 ppm or less than 1 ppm (by weight) sulfur atoms in sulfhydryl compounds. In other embodiments, the alcohol or purified alcohol has less than 10 ppm or less than 1 ppm sulfur atoms (in any form).

The methods of the invention can also involve contacting the biogas with a sulfur scrubber separate from the Fe²⁺ produced by the iron-reducing organism. The sulfur scrubber may remove one or more of sulfhydryls, H₂S, anionic oxidized forms of sulfur (e.g., sulfate and sulfite), and COS.

Sulfhydryls and other forms of sulfur can also be removed from the alcohol after it is formed. Thus, one embodiment of the invention provides a method of producing alcohol involving (a) fermenting organic material in a fermentation mixture to a biogas comprising methane; (b) converting at least a portion of the biogas to synthesis gas comprising CO and H₂; (c) contacting at least a portion of the synthesis gas with a catalyst to produce alcohol; (d) contacting the alcohol with a scrubber to remove sulfhydryls from the alcohol; and (e) purifying the alcohol, wherein the purified alcohol contains less than 10 ppm sulfur atoms, less than 5% methanol, and at least 70% C₂+ alcohols by weight.

In one embodiment, the method further involves removing sulfhydyls from the biogas before it is reformed to syngas. This can be done by adding a source of Fe³⁺ to the fermentation mixture where the fermentation mixture contains a microorganism that reduces Fe³⁺ and produces at least one volatile organic acid. Where the microorganism reduces Fe³⁺ to Fe²⁺ the Fe²⁺ binds sulfhydyls and removes them from the biogas. Preferably the microorganism is ATCC 55339 or is derived from ATCC 55339.

Sulfhydyls can also be removed from the biogas by a scrubber. The scrubber may be, for instance amino groups immobilized on a resin. This can be in addition to or as alternative to including an iron-reducing microorganism and a source of Fe³⁺ in the fermentation mixture.

In one embodiment, the step of contacting the synthesis gas with a catalyst to produce an alcohol product mixture comprises contacting the synthesis gas with a first catalyst 12a to form methanol, followed by contacting the methanol and unreacted synthesis gas with a second catalyst 12b (FIG. 2) to form C₂+ alcohols. The unreacted synthesis gas may be synthesis gas that contacted the first catalyst without reacting, or it may be a portion of the synthesis gas that bypasses the first catalyst to be taken directly to the second catalyst for reaction with methanol.

Suitable first catalysts for methanol synthesis are the MK-101 and MK-121 catalysts from Haldor Topsoe (Houston, Tex. and Lyngby, Denmark). These are sulfur-free catalysts.

Suitable second catalysts for ethanol and higher alcohol synthesis include the catalysts described in Bao, J. et al., 2003, Chem. Commun. 2003:746-747; U.S. Pat. No. 4,235,801; and U.S. Pat. No. 4,333,852. These are also sulfur-free catalysts.

In particular embodiments, the first and second catalysts are both sulfur-free.

In other embodiments, the liquid fuel is or comprises diesel fuel. In some embodiments, the method comprises purifying diesel fuel from the liquid fuel product mixture, wherein at least 90% by weight of reduced carbon compounds in the purified diesel boil between 150° C. and 350° C.

In specific embodiments, at least 50% by weight of reduced carbon compounds in the liquid fuel product mixture boil between 150° C. and 350° C.

Purifying diesel fuel from the liquid fuel product mixture can comprise fractionating the liquid fuel product mixture into a purified diesel fraction, a water-enriched fraction, a synthesis gas-enriched fraction optionally containing CO₂, and optionally a separate CO₂-enriched fraction; wherein the synthesis gas-enriched fraction is recycled to the liquid fuel-production catalyst and the CO₂-enriched fraction, if present, is recycled to one or more of the fermentation mixture, the CO₂ reforming reaction, and the liquid fuel-production catalyst.

The CO₂ from the biogas can also be fed into the liquid fuel catalysts with the syngas. CO₂ can be reduced there by H₂ to methanol and other liquid fuels. This is another means to capture the carbon atoms of CO₂ and capture as much energy as possible from the biogas.

All references cited are hereby incorporated by reference.

What is claimed is:

1. A method of producing liquid fuel comprising:
(a) cultivating aquatic plants in an aquatic medium exposed to light in a photosynthetic culture chamber;
(b) transferring organic material of the aquatic plants to an anaerobic digester;
(c) fermenting the organic material in the digester to produce a biogas comprising methane and carbon dioxide;
(d) converting at least a portion of the biogas to synthesis gas comprising CO and H₂; and
(e) contacting at least a portion of the synthesis gas with a catalyst to produce a liquid fuel.

2. The method of claim 1 wherein the step of fermenting produces an effluent; and the method further comprises transferring at least a portion of the effluent from the anaerobic digester to the photosynthetic culture chamber.

3. The method of claim 2 wherein the effluent is a liquid effluent that has a concentration of dissolved CO₂ of at least 3 mM when contacted with the aquatic medium in the photosynthetic culture chamber.

4. The method of claim 2 wherein the step of transferring the effluent from the anaerobic digester to the photosynthetic culture chamber comprises transferring the effluent from the anaerobic digester to a treatment chamber, treating the effluent in the treatment chamber to generate a treated effluent, and transferring the treated effluent to the photosynthetic culture chamber.

5. The method of claim 1 further comprising:
feeding a CO₂-rich anaerobic exhaust gas from a hydrocarbon fuel combustion process to the photosynthetic culture chamber.

6. The method of claim 1 further comprising:
separating CO₂ from the biogas; and
delivering separated CO₂ from the biogas to the aquatic medium in the photosynthetic culture chamber to enrich the aquatic medium in CO₂.

7. The method of claim 1 further comprising
contacting at least a portion of the biogas with an aqueous volume outside of the anaerobic digester and the photosynthetic culture chamber to dissolve CO₂ from the biogas in the aqueous volume and generate a CO₂-depleted biogas; and
mixing the aqueous volume with the aqueous medium in the photosynthetic culture chamber;
wherein step (d) comprises converting at least a portion of the CO₂-depleted biogas to synthesis gas.

8. The method of claim 1 further comprising:
contacting at least a portion of the biogas with the aquatic medium in the photosynthetic culture chamber to dissolve CO₂ from the biogas in the aquatic medium and generate a CO₂-depleted biogas;
wherein step (d) comprises converting at least a portion of the CO₂-depleted biogas to synthesis gas.

9. The method of claim 1 wherein the aquatic plants are planktonic and the step of transferring organic material of the aquatic plants to an anaerobic digester comprises transferring the aquatic medium containing the aquatic plants to the digester, without separating the aquatic plants from the aquatic medium.

10. The method of claim 1 further comprising between steps (a) and (b) harvesting the aquatic plants from the aquatic medium.

11. The method of claim 1 further comprising between steps (a) and (b) processing the aquatic plants to isolate oil and organic residue from the aquatic plants; step (b) comprises transferring the organic residue to an anaerobic digester; and step (c) comprises fermenting the organic residue in the digester.

12. The method of claim 1 wherein the photosynthetic culture chamber is positioned above the anaerobic digester.

13. The method of claim 1 wherein the liquid fuel comprises alcohol or diesel fuel.

14. The method of claim 1 wherein the aquatic plants are duckweed or water hyacinth.

15. The method of claim 1 wherein the aquatic plants are macroalgae or microalgae.

16. A method of producing biogas comprising:
(a) cultivating aquatic plants in an aquatic medium exposed to light in a photosynthetic culture chamber;
(b) transferring organic material of the aquatic plants to an anaerobic digester;
(c) fermenting the organic material in the digester to produce a liquid effluent and a biogas comprising methane; and
(d) transferring CO₂ to the aquatic medium in the photosynthetic culture chamber by
(i) transferring at least a portion of the liquid effluent from the anaerobic digester to the photosynthetic culture chamber wherein the liquid effluent has a concentration of dissolved CO₂ of at least 3 mM when contacted with the aquatic medium in the photosynthetic culture chamber; or
(ii) contacting at least a portion of the biogas with an aqueous volume outside of the anaerobic digester and the photosynthetic culture chamber to dissolve CO₂ from the biogas in the aqueous volume and generate a CO₂-depleted biogas, and mixing the aqueous volume with the aquatic medium in the photosynthetic culture chamber; or
(iii) contacting at least a portion of the biogas with the aquatic medium in the photosynthetic culture chamber to dissolve CO₂ from the biogas in the aquatic medium and generate a CO₂-depleted biogas.

17. A system for producing liquid fuel comprising:
(a) a photosynthetic culture chamber for culturing aquatic plants in an aquatic medium exposed to light in the photosynthetic culture chamber; (a) being functionally coupled to
(b) an anaerobic digester for producing biogas comprising CH₄ and CO₂ from organic material of the aquatic plants in an anaerobic digestion mixture in the anaerobic digester; (b) being functionally coupled to
(c) a device for producing synthesis gas comprising CO and H₂ from biogas comprising CH₄, the device comprising a steam reformer, a CO₂ reformer, a partial oxidation unit, or a combination thereof; (c) being functionally coupled to
(d) a catalyst for converting synthesis gas to a liquid fuel.

18. A system for producing biogas comprising:
(a) a photosynthetic culture chamber for culturing aquatic plants in an aquatic medium exposed to light in the photosynthetic culture chamber, (a) being functionally coupled to
(b) an anaerobic digester for producing biogas comprising CH₄ and CO₂ from organic material of the aquatic plants in an anaerobic digestion mixture in the anaerobic digester; (b) being functionally coupled to (c)
(c) a conduit functionally coupled to (a) and (b) and adapted for transferring liquid effluent from the anaerobic digester to the aquatic medium in the photosynthetic culture chamber without equilibration with air before the liquid effluent is mixed with the aquatic medium in the photosynthetic culture chamber.

19. A system for producing biogas comprising:
(a) a photosynthetic culture chamber for culturing aquatic plants in an aquatic medium exposed to light in the photosynthetic culture chamber; (a) being functionally coupled to
(b) an anaerobic digester for producing biogas comprising CH₄ and CO₂ from organic material of the aquatic plants in an anaerobic digestion mixture in the anaerobic digester;
(c) a chamber separate from the anaerobic digester and photosynthetic culture chamber for holding an aqueous volume;
(d) a conduit functionally coupled to (b) and (c) and adapted to transfer biogas from the anaerobic digester (b) to (c) and contact the biogas with the aqueous volume in the chamber (c) to dissolve CO₂ from the biogas in the aqueous volume in (c), the chamber (c) functionally coupled to
(e) a conduit functionally coupled to (c) and (a) and adapted to transfer the aqueous volume to aquatic medium in the photosynthetic culture chamber without equilibration with air before the aqueous volume is mixed with the aquatic medium in the photosynthetic culture chamber.

20. A system for producing biogas comprising:
(a) a photosynthetic culture chamber for culturing aquatic plants in an aquatic medium exposed to light in the photosynthetic culture chamber; (a) being functionally coupled to
(b) an anaerobic digester for producing biogas comprising CH₄ and CO₂ from organic material of the aquatic plants in an anaerobic digestion mixture in the anaerobic digester; (b) being functionally coupled to
(c) a conduit functionally coupled to the anaerobic digester and the photosynthetic culture chamber and adapted to transfer at least a portion of the biogas from the anaerobic digester to the photosynthetic culture chamber to contact the at least a portion of the biogas with the aquatic medium in the photosynthetic culture chamber to dissolve CO₂ from the biogas in the aquatic medium and generate a CO₂-depleted biogas.
